(19) United States
(12) Patent Application Publication
(10) Pub. No.: US 2024/0209312 A1
(43) Pub. Date: Jun. 27, 2024

(54) Hydrogen-Oxidizing Bacteria Culturing Method And Hydrogen-Oxidizing Bacteria Culturing Apparatus

(71) Applicant: SEIKO EPSON CORPORATION, Tokyo (JP)

(72) Inventors: Yasutoshi HIDESHIMA, Matsumoto (JP); Fumiya MAEDA, Matsumoto (JP)

(21) Appl. No.: 18/393,794

(22) Filed: Dec. 22, 2023

(30) Foreign Application Priority Data
Dec. 26, 2022 (JP) 2022-207949

(51) Int. Cl. C12N 1/20; C01B 3/08; C12M 1/00

(52) U.S. Cl. CPC C12N 1/20; C01B 3/08; C12M 29/04; C12M 29/06

(57) ABSTRACT

A method includes: supplying a gas 600 containing carbon dioxide to a liquid surface 201 of a liquid medium 200 inoculated with hydrogen-oxidizing bacteria that are growable using the carbon dioxide as a carbon source; and supplying hydrogen 700 to the hydrogen-oxidizing bacteria, the hydrogen being generated by bringing the liquid medium 200 into contact with a metal body 300 and causing a corrosion reaction in the metal body 300.

The present application is based on, and claims priority from JP Application Serial Number 2022-207949, filed Dec. 26, 2022, the disclosure of which is hereby incorporated by reference herein in its entirety.

### BACKGROUND

### 1. Technical Field

The present disclosure relates to a hydrogen-oxidizing bacteria culturing method and a hydrogen-oxidizing bacteria culturing apparatus.

### 2. Related Art

Japanese Patent No. 6450912 discloses, as a hydrogen-oxidizing bacteria culturing method, a method in which a medium is first placed in a culture vessel, then, a mixed gas containing hydrogen, oxygen, and carbon dioxide is supplied into the culture vessel, and hydrogen-oxidizing bacteria are subjected to static culture or shaking culture in the culture vessel to which the mixed gas is supplied. Japanese Patent No. 6450912 also discloses that, by optimizing a volume ratio among hydrogen, oxygen, and carbon dioxide in the mixed gas, the growth of the hydrogen-oxidizing bacteria gets better, and a target compound can be efficiently produced.

However, in the culture method described in Japanese Patent No. 6450912, it is necessary to prepare a large amount of hydrogen, oxygen, and carbon dioxide in advance. In particular, since hydrogen is a combustible gas, it is necessary to pay close attention to the storage of hydrogen. Therefore, the culture method described in Japanese Patent No. 6450912 has problems in that, for example, safety is necessary to be considered and the burden of equipment investment is large. In addition, in order to supply the mixed gas into the culture vessel, it is necessary to store the mixed gas under pressurization or use a pump. Therefore, in consideration of social implementation of the hydrogen-oxidizing bacteria, it is necessary to reduce energy required for the storage and the supply of hydrogen.

### SUMMARY

A hydrogen-oxidizing bacteria culturing method includes: supplying a gas containing carbon dioxide to a liquid surface of a liquid medium inoculated with hydrogen-oxidizing bacteria that are growable using the carbon dioxide as a carbon source; and supplying hydrogen to the hydrogen-oxidizing bacteria, the hydrogen being generated by bringing the liquid medium into contact with a metal body and causing a corrosion reaction in the metal body.

A hydrogen-oxidizing bacteria culturing apparatus includes: a culture vessel; a liquid medium accommodated in the culture vessel and to be inoculated with hydrogen-oxidizing bacteria; a metal body configured to generate hydrogen by causing a corrosion reaction by contact with the liquid medium; and a nozzle disposed to face a liquid surface of the liquid medium and configured to supply a gas containing carbon dioxide to the liquid surface.

### BRIEF DESCRIPTION OF THE DRAWINGS

FIG. 1 is a diagram schematically showing a configuration of hydrogen-oxidizing bacteria.

FIG. 2 is a cross-sectional view showing a configuration of a hydrogen-oxidizing bacteria culturing apparatus according to a first embodiment.

FIG. 3 is a perspective view showing the configuration of the hydrogen-oxidizing bacteria culturing apparatus.

FIG. 4 is a plan view showing the configuration of the hydrogen-oxidizing bacteria culturing apparatus.

FIG. 5 is a perspective view showing a configuration of a nozzle of the culturing apparatus.

FIG. 6 is a flowchart showing a hydrogen-oxidizing bacteria culturing method.

FIG. 7 is a diagram showing some steps of the hydrogen-oxidizing bacteria culturing method.

FIG. 8 is a cross-sectional view showing a configuration of a hydrogen-oxidizing bacteria culturing apparatus according to a second embodiment.

FIG. 9 is a flowchart showing a hydrogen-oxidizing bacteria culturing method.

FIG. 10 is a plan view showing a configuration of a nozzle according to a modification.

FIG. 11 is a plan view showing a configuration of a nozzle according to a modification.

FIG. 12 is a perspective view showing a configuration of a hydrogen-oxidizing bacteria culturing apparatus according to a modification.

FIG. 13 is a perspective view showing a configuration of a hydrogen-oxidizing bacteria culturing apparatus according to a modification.

FIG. 14 is a perspective view showing a configuration of a hydrogen-oxidizing bacteria culturing apparatus according to a modification.

FIG. 15 is a perspective view showing a configuration of a hydrogen-oxidizing bacteria culturing apparatus according to a modification.

FIG. 16 is a perspective view showing a configuration of a hydrogen-oxidizing bacteria culturing apparatus according to a modification.

FIG. 17 is a plan view showing a configuration of a nozzle according to a modification.

FIG. 18 is a plan view showing a configuration of a nozzle according to a modification.

FIG. 19 is a plan view showing a configuration of a nozzle according to a modification.

FIG. 20 is a plan view showing a configuration of a nozzle according to a modification.

FIG. 21 is a perspective view showing a configuration of a hydrogen-oxidizing bacteria culturing apparatus according to a modification.

FIG. 22 is a cross-sectional view showing a configuration of a hydrogen-oxidizing bacteria culturing apparatus according to a modification.

FIG. 23 is a cross-sectional view showing a configuration of a hydrogen-oxidizing bacteria culturing apparatus according to a modification.

FIG. 24 is a cross-sectional view showing a configuration of a hydrogen-oxidizing bacteria culturing apparatus according to a modification.

FIG. 25 is a cross-sectional view showing a configuration of a hydrogen-oxidizing bacteria culturing apparatus according to a modification.

FIG. 26 is a cross-sectional view showing a configuration of a hydrogen-oxidizing bacteria culturing apparatus according to a modification.

FIG. 27 is a cross-sectional view showing a configuration of a hydrogen-oxidizing bacteria culturing apparatus according to a modification.

FIG. 28 is a cross-sectional view showing a configuration of a hydrogen-oxidizing bacteria culturing apparatus according to a modification.

FIG. 29 is a cross-sectional view showing a configuration of a hydrogen-oxidizing bacteria culturing apparatus according to a modification.

FIG. 30 is a cross-sectional view showing a configuration of a hydrogen-oxidizing bacteria culturing apparatus according to a modification.

FIG. 31 is a cross-sectional view showing a configuration of a hydrogen-oxidizing bacteria culturing apparatus according to a modification.

### DESCRIPTION OF EMBODIMENTS

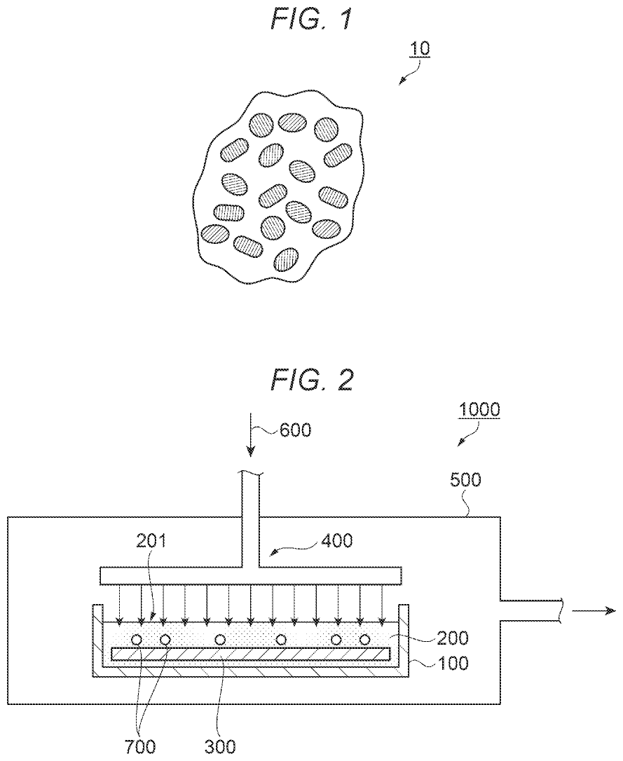

First, hydrogen-oxidizing bacteria 10 will be described with reference to FIG. 1.

As shown in FIG. 1, the hydrogen-oxidizing bacteria 10 are bacteria that can grow using hydrogen as an energy source and can grow using carbon dioxide as a carbon source. A chemical product can be synthesized by using the hydrogen-oxidizing bacteria 10, hydrogen, and carbon dioxide. Accordingly, carbon dioxide, which is a greenhouse gas, can be used as a resource, and thus the implementation of carbon neutrality can be promoted.

In order to synthesize a chemical product in this manner, it is required to easily and efficiently culture the hydrogen-oxidizing bacteria 10. In particular, when a large amount of a hydrogen gas is stored and transported, the time and effort and cost are necessary to meet various restrictions on safety. Therefore, it is required to reduce the amount of the hydrogen gas stored and transported as much as possible. In addition, compression or liquefaction of the hydrogen gas is often required for the storage or the transportation. A large amount of energy is consumed for the compression or liquefaction of the hydrogen gas, and therefore, a reduction in energy consumption is also a problem in culture of the hydrogen-oxidizing bacteria 10.

### FIRST EMBODIMENT

Next, a configuration of a culturing apparatus 1000 for the hydrogen-oxidizing bacteria 10 according to a first embodiment will be described with reference to FIGS. 2 to 5.

As shown in FIG. 2, the culturing apparatus 1000 for the hydrogen-oxidizing bacteria 10 includes a culture vessel 100, a liquid medium 200 accommodated in the culture vessel 100, a metal body 300 disposed in the liquid medium 200, a nozzle 400 disposed to face a liquid surface 201 of the liquid medium 200, and a chamber 500 in a closed state, which accommodates the culture vessel 100, the nozzle 400, and the like.

As described above, the hydrogen-oxidizing bacteria 10 grow by hydrogen 700 and oxygen supplied to the liquid medium 200, and implement carbon dioxide fixation. As a result, various chemical products are produced. The hydrogen-oxidizing bacteria 10 are not particularly limited as long as they can grow using hydrogen as an energy source and carbon dioxide as a carbon source.

A material for the culture vessel 100 is not particularly limited as long as it does not react with the liquid medium 200 or does not degenerate due to the hydrogen 700. Examples of the material include stainless steel. The material is not limited to stainless steel, and may be a metal material, a glass material, a ceramic material, a resin material, or the like.

The liquid medium 200 is a liquid which contains water and in which the hydrogen-oxidizing bacteria 10 are inoculated. Specifically, the liquid medium 200 is, for example, a liquid medium obtained by dispersing an organic medium or an inorganic medium in water. The organic medium is, for example, a medium containing organic substances such as sugars, organic acids, and amino acids. Examples of the inorganic medium include a medium containing a carbonate. The metal body 300 generates the hydrogen (H₂) 700 by causing a corrosion reaction by the contact with the liquid medium 200.

The nozzle 400 supplies a gas 600 containing carbon dioxide (CO₂) to the liquid surface 201 of the liquid medium 200. Carbon dioxide is a carbon source necessary for production of chemical products. The gas 600 may contain oxygen (O₂). As shown in FIG. 5, the nozzle 400 has a plurality of opening holes 410. FIG. 5 is a plan view of the nozzle 400 as viewed from a culture vessel 100 side. The nozzle 400 has the plurality of opening holes 410, so that the gas 600 can be blown to a wide range on the liquid surface 201.

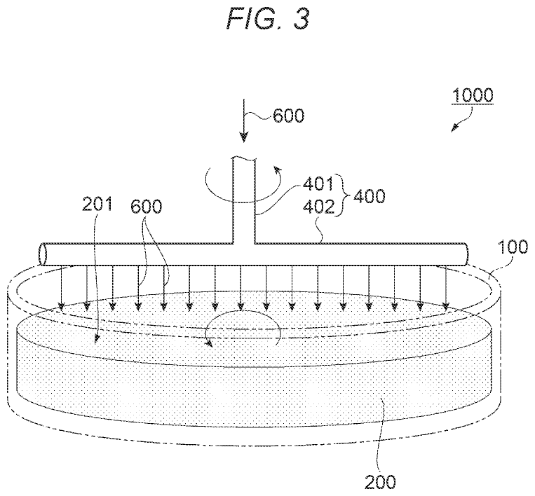

As shown in FIG. 3, the nozzle 400 includes a first pipe 401 extending in a vertical direction and taking in the gas 600, and a second pipe 402 extending in a horizontal direction from an end portion of the first pipe 401 and provided with the plurality of opening holes 410.

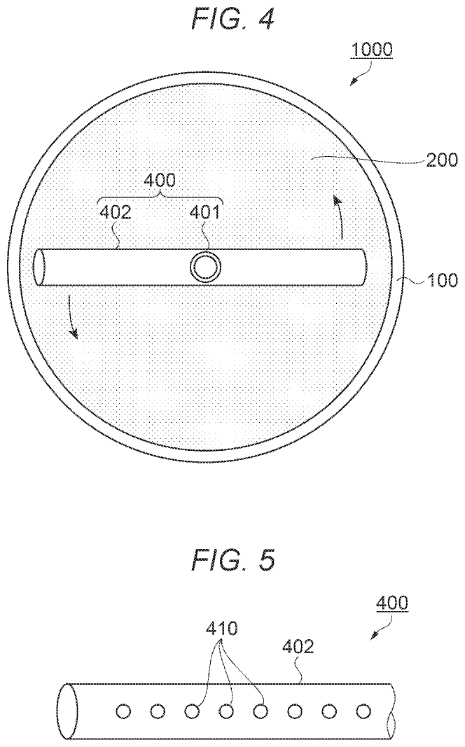

As shown in FIGS. 3 and 4, the nozzle 400 is provided such that the second pipe 402 rotates about the first pipe 401. The gas 600 can be blown to the entire liquid surface 201 of the liquid medium 200 in the culture vessel 100 by rotating the second pipe 402.

As shown in FIG. 3, the culture vessel 100 rotates in a direction opposite to a rotation direction of the nozzle 400. In other words, it is preferable that the culture vessel 100 and the nozzle 400 are moved relative to each other. The rotation speeds of the culture vessel 100 and the nozzle 400 are, for example, constant or intermittent.

As described above, according to the culturing apparatus 1000, the culture vessel 100 and the nozzle 400 are moved relative to each other, and therefore, the gas 600 can be blown onto the liquid surface 201 of the liquid medium 200 to ripple the liquid surface 201.

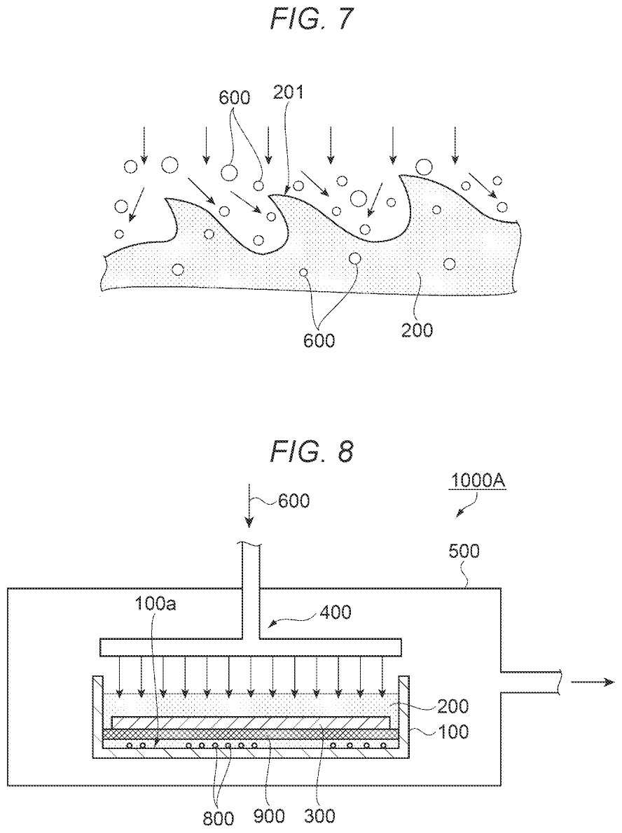

The liquid surface 201 is rippled (see FIG. 7), so that the gas 600 can be efficiently brought into contact with the liquid surface 201 whose surface area is increased by rippling, that is, the liquid medium 200. Accordingly, the gas 600 can be efficiently mixed in the liquid medium 200, and the hydrogen-oxidizing bacteria 10 can be cultured.

It should be noted that the pressure at which the rippling is enabled refers to a state in which an interface between the liquid medium 200 and the blown gas 600 moves strongly as shown in FIG. 7, for example. Specifically, the state of a severe movement can be determined by actually measuring the size of the gas 600 in the form of bubbles. For example, when the size of the bubble is about 10 μm to 50 μm, the bubble having this size is called a microbubble, and when the size of the bubble is 100 nm or less, the bubble having this size can be called a nanobubble. With a state in which such air bubbles are generated, it can be determined that the liquid surface 201 is rippled.

In addition, a pressure for blowing the gas 600 can be expressed as (gas-liquid contact area when the gas is blown)÷(gas-liquid contact area when the gas is not blown)≥1.1. As the measurement method, simulation or a high speed camera is used.

The intervals between the opening holes 410 of the nozzle 400 shown in FIG. 5 may be uniform or non-uniform. In addition, the sizes of the opening holes 410 may be all the same as or different from each other. It is preferable that the sizes of the opening holes 410 and the intervals therebetween appropriately cope with each other in accordance with the rippling condition of the liquid surface 201 when the gas 600 is blown onto the liquid surface 201.

According to the culturing apparatus 1000 for the hydrogen-oxidizing bacteria 10, the hydrogen 700 can be easily generated by the contact between the metal body 300 and the liquid medium 200. Therefore, the hydrogen 700 can be supplied to the hydrogen-oxidizing bacteria 10 inoculated in the liquid medium 200. Accordingly, the hydrogen 700 can be supplied to the liquid medium 200 without storing or transporting a large amount of the hydrogen 700. Since it is not necessary to store the hydrogen 700, consumption of energy necessary for supplying the hydrogen 700 can be reduced. The liquid medium 200 is stirred, and therefore, a reaction rate of corrosion of the metal body 300 can be further increased.

As for a volume ratio of each gas necessary for culturing the hydrogen-oxidizing bacteria 10, a ratio of hydrogen:oxygen:carbon dioxide is about 8:1:1 as an example. The volume ratio may be out of this ratio, and basically, the volume ratio of the hydrogen 700 is the largest. Therefore, a flow rate of the gas 600 may be sufficiently smaller than that of the hydrogen 700, and the energy necessary for the supply can also be reduced to be sufficiently small.

A material for the metal body 300 is not limited as long as it can corrode upon the contact with the liquid medium 200 to generate the hydrogen 700. Examples thereof include magnesium (Mg). The metal body 300 preferably contains a metal element having an ionization tendency higher than that of the hydrogen 700. Accordingly, the metal body 300 can efficiently generate the hydrogen 700 by contact with the liquid medium 200.

Examples of the metal element having an ionization tendency higher than that of the hydrogen 700 include Li, K, Ca, Na, Mg, Al, Ti, Zn, Fe, Co, Ni, Sn, and Pb. Among them, Ca, Mg, Al, Ti, or Zn is preferably used in consideration of handleability, hydrogen generation efficiency, and the like.

As a constituent material of the metal body 300, an elemental metal or a compound containing these metal elements is preferably used, and in particular, an elemental metal, a hydrogenated compound composed of these metal elements or the like is more preferably used. When the constituent material is an elemental metal or a hydrogenated compound, the metal body 300 having particularly good reactivity with the liquid medium 200 can be implemented.

Specific examples of the constituent material of the metal body 300 include elemental calcium, a calcium-based alloy, calcium hydride, elemental magnesium, a magnesium-based alloy, magnesium hydride, elemental aluminum, an aluminum-based alloy, aluminum hydride, or composite materials containing them.

Among them, elemental magnesium, a magnesium-based alloy, magnesium hydride, or a composite material containing any one thereof is preferably used, and in particular, a magnesium-based alloy or a composite material containing a magnesium-based alloy is more preferably used. They are useful as the constituent material of the metal body 300 because the hydrogen generation efficiency is particularly high.

Examples of the magnesium-based alloy include AZ91A, AZ91B, AZ91D, AM60A, AM60B, AS41A, AZ31, AZ31B, AZ61A, AZ63A, AZ80A, AZ91C, AZ91E, AZ92A, AM100A, ZK51A, ZK60A, ZK61A, EZ33A, QE22A, ZE41A, M1A, WE54A, and WE43B of standards of the American Society for Testing and Materials (ASTM).

On the other hand, examples of the composite material include a composite material of a material containing the above-described metal element and a material containing a component that promotes corrosion. The former material constitutes, for example, a matrix portion of the metal body 300, and the latter material constitutes particles dispersed in the matrix portion. With such a composite material, when the metal body 300 comes into contact with the liquid medium 200, a local cell is formed between the matrix portion and the particles, and the local cell is evenly formed over the entire metal body 300. Accordingly, a corrosion reaction can be caused at a high rate, and a decrease in reaction rate due to a corrosion product 800 (see FIG. 8) as a by-product is easily prevented.

Examples of the form of the metal body 300 include a powder form, a granule form, a block form, a chip form, a plate form, a rod form, and a linear form. A molded body having a more complicated shape may be formed by molding. A specific surface area changes depending on the form of the metal body 300. The specific surface area of the metal body 300 influences a generation rate of the hydrogen 700, and therefore, the form of the metal body 300 may be selected according to the desired generation rate of hydrogen.

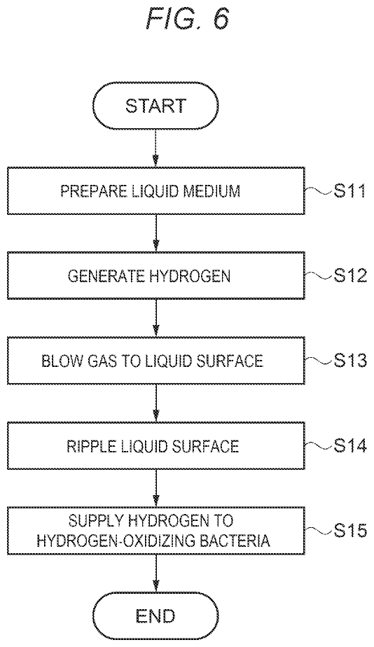

Next, a method for culturing the hydrogen-oxidizing bacteria 10 will be described with reference to FIGS. 6 and 7.

As shown in FIG. 6, the liquid medium 200 is prepared in step S11. Specifically, the hydrogen-oxidizing bacteria 10 that can grow using carbon dioxide as a carbon source are inoculated in the liquid medium 200.

In step S12, the hydrogen 700 is generated from the metal body 300. Specifically, the metal body 300 is charged into the liquid medium 200 at a timing at which the hydrogen 700 is desired to be generated. The metal body 300 can be charged, for example, through an opening (not shown) provided in the chamber 500. Accordingly, a corrosion reaction is caused in the metal body 300 by the contact with the liquid medium 200 to generate the hydrogen 700. In this way, the hydrogen 700 can be generated at a necessary timing without storing or transporting a large amount of the hydrogen 700.

When the metal body 300 comes into contact with the liquid medium 200, a corrosion reaction is caused in the metal body 300 to generate the hydrogen 700. As described above, the metal body 300 preferably contains a metal element having an ionization tendency higher than that of the hydrogen 700. Accordingly, the metal body 300 can efficiently generate the hydrogen 700 by the contact with the liquid medium 200.

The hydrogen 700 is a gas generated by a corrosion reaction in the metal body 300 in the culture vessel 100. The hydrogen 700 is converted into bubbles from the metal body 300, and is supplied to the hydrogen-oxidizing bacteria 10.

The hydrogen 700 can be supplied by transporting and using a gas produced by other methods or by generation by electrolysis of water. There is a risk of explosion, and thus care must be taken when handling. On the other hand, carbon dioxide is a gas that is not intended to be emitted, and there are difficulties in the treatment of carbon dioxide. That is, the hydrogen 700 can be easily supplied, and a social useful system is obtained by using unnecessary carbon dioxide.

In the step of generating the hydrogen 700, an operation of adjusting an amount of hydrogen generated from the metal body 300 per unit time may be performed. Examples of such an operation include an operation of changing at least one of a temperature and pH of the liquid medium 200, a flow rate of the liquid medium 200, and a moving speed of the metal body 300 relative to the liquid medium 200. The hydrogen generation amount can be adjusted to a target value by including such an operation. Accordingly, the amount of hydrogen to be wasted can be reduced, and the corrosion of the metal body 300 can be maintained for a longer time. As a result, the consumption efficiency of the metal body 300 can be increased.

In step S13, the gas 600 containing carbon dioxide is supplied, that is, blown to the liquid surface 201 of the liquid medium 200 in the culturing apparatus 1000. The gas 600 is carbon dioxide (CO₂) or a mixed gas of carbon dioxide (CO₂) and oxygen (O₂).

The pressure for blowing the gas 600 varies depending on a viscosity of the liquid medium 200, and is required to be a pressure at which the liquid medium 200 does not start to scatter. The pressure is adjusted as needed depending on a degree of proliferation of the hydrogen-oxidizing bacteria 10.

Next, in step S14, the liquid surface 201 of the liquid medium 200 is rippled. Specifically, the gas 600 is blown from the opening holes 410 of the nozzle 400 to portions corresponding to at least a plurality of locations of the liquid surface 201. The pressure of the gas 600 to be blown is a pressure at which the liquid surface 201 is rippled. The second pipe 402 of the nozzle 400 and the culture vessel 100 are moved relative to each other. Accordingly, as shown in FIG. 7, the liquid surface 201 can be rippled, and the gas 600 can be supplied into the rippled liquid medium 200.

In this way, by rippling the liquid surface 201 of the liquid medium 200 with the pressure of the gas 600, the stable hydrogen-oxidizing bacteria 10 can be proliferated, and the energy loss associated therewith can be reduced.

Next, in step S15, the hydrogen 700 and the gas 600 are supplied to the hydrogen-oxidizing bacteria 10. When the liquid surface 201 is rippled, the gas 600 can be efficiently brought into contact with the liquid medium 200 whose surface area is increased by rippling. Therefore, the gas 600 can be efficiently mixed into the liquid medium 200. Accordingly, the hydrogen 700 and the gas 600 can be supplied to the hydrogen-oxidizing bacteria 10 in the liquid medium 200.

Accordingly, the hydrogen-oxidizing bacteria 10 grow to implement the carbon dioxide fixation. As a result, various chemical products are produced. For the liquid medium 200, the water quality of the liquid medium 200, such as a temperature, pH, dissolved hydrogen, and dissolved oxygen thereof, may be monitored, an exhaust gas component in the liquid medium 200 may be monitored, and the measurement results are fed back to adjust the water quality of the liquid medium 200, such as the temperature and pH thereof, as appropriate.

By performing such an operation, the hydrogen generation amount can be adjusted to a target value, and the amount of hydrogen to be wasted can be reduced. Further, the corrosion of the metal body 300 can be maintained for a longer time, and as a result, the consumption efficiency of the metal body can be increased.

According to the culturing apparatus 1000 for the hydrogen-oxidizing bacteria 10 having such a configuration, the hydrogen 700 having a high concentration is not necessary for the mixed gas, and it is possible to cope with the mixed gas only by handling the carbon dioxide gas and the oxygen gas which are relatively safe. Note that the excess hydrogen 700 can be collected together with the excess of the carbon dioxide gas and the oxygen gas, and can be charged into the culturing apparatus 1000 again. In addition, an amount of the excess hydrogen 700 can be controlled by a charging amount of the metal body 300 or the like, and can be constantly controlled to be equal to or lower than an explosion limit.

According to such a method for culturing the hydrogen-oxidizing bacteria 10, hydrogen can be generated at a necessary timing without storing or transporting a large amount of hydrogen. In addition, since the supply of the hydrogen 700 is not accompanied by compression or liquefaction of the hydrogen at a high pressure, energy consumption in the supply of the hydrogen can be reduced. Accordingly, the hydrogen-oxidizing bacteria 10 can be efficiently cultured at a low cost while reducing equipment investment. As a result, a chemical product can be produced while contributing to the carbon neutrality by implementing the carbon dioxide fixation.

As described above, the hydrogen-oxidizing bacteria 10 can be cultured. The cultured hydrogen-oxidizing bacteria 10 contain the produced chemical products, and therefore, the produced chemical products may be collected by any collection method. Examples of the collection method include various separation methods such as fractionation, extraction, ultrasonic atomization separation, chromatography, and crystallization. The chemical products to be produced are not particularly limited, and examples thereof include ethanol, isobutanol, and lactic acid. The cultured hydrogen-oxidizing bacteria themselves can be used as chemical products. Specifically, the cultured hydrogen-oxidizing bacteria 10 can be used as various feeds such as livestock feeds and fish culture feeds, protein resources, and the like.

As described above, the method for culturing the hydrogen-oxidizing bacteria 10 according to the present embodiment includes: supplying the gas 600 containing carbon dioxide to the liquid surface 201 of the liquid medium 200 inoculated with the hydrogen-oxidizing bacteria 10 that can grow using carbon dioxide as a carbon source; and supplying the hydrogen 700 to the hydrogen-oxidizing bacteria 10, the hydrogen 700 being generated by bringing the liquid medium 200 into contact with the metal body 300 and causing a corrosion reaction in the metal body 300.

According to this method, the gas 600 can be mixed in the liquid medium 200 since the gas 600 is supplied to the liquid surface 201 of the liquid medium 200, in other words, the gas 600 is brought into contact with the liquid surface 201. Therefore, carbon dioxide necessary for growth can be supplied to the hydrogen-oxidizing bacteria 10 in the liquid medium 200. As a result, a large amount of hydrogen-oxidizing bacteria 10 can be efficiently cultured.

In the step of supplying the gas 600 in the method for culturing the hydrogen-oxidizing bacteria 10 according to the present embodiment, it is preferable to blow the gas 600 at a pressure at which the liquid surface 201 of the liquid medium 200 can be rippled. According to this method, the gas 600 is blown to the liquid surface 201 at the above-described pressure, and therefore, the liquid surface 201 can be rippled, and the gas 600 can be efficiently brought into contact with the liquid medium 200 whose surface area is increased by rippling. Therefore, the supply efficiency of the liquid medium 200 can be improved. The gas 600 can be supplied into the liquid medium 200, and the liquid medium 200 can be stirred by blowing the gas 600 into the liquid medium 200.

In the step of supplying the gas 600 in the method for culturing the hydrogen-oxidizing bacteria 10 according to the present embodiment, it is preferable to blow the gas 600 to portions corresponding to at least a plurality of locations of the liquid surface 201. According to this method, the gas 600 is blown to the plurality of locations of the liquid surface 201, and therefore, the liquid surface 201 can be rippled, and the gas 600 can be supplied into the rippled liquid medium 200.

In the step of supplying the gas 600 in the method for culturing the hydrogen-oxidizing bacteria 10 according to the present embodiment, it is preferable that the liquid medium 200 and the supplied gas 600 are moved relative to each other. According to this method, the liquid medium 200 and the gas 600 to be supplied are moved relative to each other, and therefore, the liquid surface 201 can be rippled and the gas 600 can be supplied into the rippled liquid medium 200.

In the method for culturing the hydrogen-oxidizing bacteria 10 according to the present embodiment, the metal body 300 preferably contains a metal element having an ionization tendency higher than that of the hydrogen 700. According to this method, the metal body 300 contains the metal element, and therefore, the hydrogen 700 can be efficiently generated by the contact with the liquid medium 200.

In the method for culturing the hydrogen-oxidizing bacteria 10 according to the present embodiment, the metal element is preferably Ca, Mg, Al, Ti, or Zn. According to this method, the above-described metal elements are used, and therefore, the handleability, hydrogen generation efficiency, and the like can be increased.

In the method for culturing the hydrogen-oxidizing bacteria 10 according to the present embodiment, the metal body 300 preferably contains a magnesium-based alloy or a composite material containing a magnesium-based alloy. According to this method, the metal body 300 is made of the above-described material, and therefore, the hydrogen generation efficiency can be increased.

In the method for culturing the hydrogen-oxidizing bacteria 10 according to the present embodiment, the step of supplying the hydrogen to the hydrogen-oxidizing bacteria preferably includes an operation of changing at least one of the temperature and pH of the liquid medium 200, the flow rate of the liquid medium 200, and the moving speed of the metal body 300 relative to the liquid medium 200. According to this method, the above-described operation is included, and therefore, the hydrogen generation amount can be adjusted to a target value, and the amount of hydrogen to be wasted can be reduced. Further, the corrosion of the metal body 300 can be maintained for a longer time, and as a result, the consumption efficiency of the metal body 300 can be increased.

In addition, the culturing apparatus 1000 for the hydrogen-oxidizing bacteria 10 according to the present embodiment includes the culture vessel 100, the liquid medium 200 accommodated in the culture vessel 100 and to be inoculated with the hydrogen-oxidizing bacteria 10, the metal body 300 which generates the hydrogen 700 by causing a corrosion reaction by the contact with the liquid medium 200, and the nozzle 400 which is disposed to face the liquid surface 201 of the liquid medium 200 and supplies the gas 600 containing carbon dioxide to the liquid surface 201.

According to this configuration, the gas 600 can be supplied to the liquid medium 200 by using the nozzle 400, in other words, the gas 600 can be blown onto the liquid surface 201 to ripple the liquid surface 201, and the gas 600 containing carbon dioxide can be supplied into the liquid medium 200 whose surface area is increased by rippling, that is, the gas 600 can be brought into contact with the liquid medium 200 whose surface area is increased, so that the gas 600 can be mixed into the liquid medium 200. Therefore, carbon dioxide necessary for growth can be efficiently supplied to the hydrogen-oxidizing bacteria 10 in the liquid medium 200.

In addition, in the culturing apparatus 1000 for the hydrogen-oxidizing bacteria 10 according to the present embodiment, it is preferable to move the culture vessel 100 and the nozzle 400 relative to each other. According to this configuration, the culture vessel 100 and the nozzle 400 are moved relative to each other, and therefore, the liquid surface 201 can be rippled by blowing the gas 600 onto the liquid surface 201 of the liquid medium 200, and the gas 600 containing carbon dioxide can be supplied into the liquid medium 200 whose surface area is increased by the rippling.

### SECOND EMBODIMENT

Next, a configuration of a culturing apparatus 1000A for the hydrogen-oxidizing bacteria 10 according to a second embodiment will be described with reference to FIG. 8.

The culturing apparatus 1000A for the hydrogen-oxidizing bacteria 10 according to the second embodiment is different from the culturing apparatus 1000 according to the first embodiment in that a product permeable body 900 composed of a net or the like that allows the corrosion product 800 to permeate therethrough is provided below the metal body 300. Therefore, in the culturing apparatus 1000A according to the second embodiment, the same components as those of the culturing apparatus 1000 according to the first embodiment are denoted by the same reference numerals, and detailed description thereof will be omitted.

As shown in FIG. 8, in the culture vessel 100 of the culturing apparatus 1000A for the hydrogen-oxidizing bacteria 10 according to the second embodiment, the product permeable body 900 is disposed at a predetermined interval from a bottom surface. The metal body 300 is placed on the product permeable body 900.

The product permeable body 900 is, for example, a net provided with holes having a size through which the corrosion product 800 generated from the metal body 300 or the like can permeate. Regarding the size of the holes, for example, when a size of the corrosion product 800 is about 1 μm, the size of the holes is preferably larger than that of the corrosion product 800. The size of the holes is preferably a size that does not allow the corrosive metal body 300 to fall from the product permeable body 900.

A state in which the metal body 300 is not in contact with a bottom portion 100a of the culture vessel 100 can be created by disposing the product permeable body 900. Accordingly, the hydrogen 700 can be generated from the entire periphery of the metal body 300, and the hydrogen 700 can be efficiently generated.

The corrosion product 800 precipitates on the bottom portion 100a of the culture vessel 100 by passing through the product permeable body 900. Therefore, even when the corrosion product 800 that inhibits the generation of the hydrogen 700 is generated, the entire surface of the metal body 300 is less likely to be covered with the corrosion product 800. As a result, the corrosion of the metal body 300 can be continued, and the hydrogen 700 can be efficiently generated.

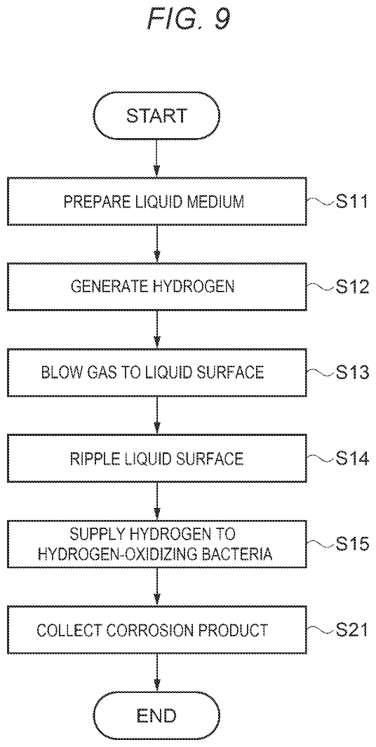

Next, a method for culturing the hydrogen-oxidizing bacteria 10 according to the second embodiment will be described with reference to FIG. 9.

As shown in FIG. 9, steps S11 to S15 are the same as those in the culture method according to the first embodiment.

In step S15, the hydrogen-oxidizing bacteria 10 grow by the hydrogen 700 and the gas 600 supplied to the hydrogen-oxidizing bacteria 10. At this time, the corrosion product 800 generated by the corrosion of the metal body 300 passes through the holes of the product permeable body 900 disposed below the metal body 300, and precipitates on the bottom portion 100a of the culture vessel 100.

Note that a powder from the metal body 300 passes through the product permeable body 900 and precipitates on the bottom portion 100a of the culture vessel 100.

Next, in step S21, the corrosion product 800 precipitated on the bottom portion 100a of the culture vessel 100 is collected. Specifically, the collection method is not particularly limited. For example, after the supply of the gas 600 is stopped, the culture vessel 100 is taken out, the metal body 300 and the product permeable body 900 are removed, and then only the corrosion product 800 is collected.

Accordingly, by collecting the corrosion product 800, the contact between the hydrogen-oxidizing bacteria 10 and the corrosion product 800 can be prevented, and the influence on the culture of the hydrogen-oxidizing bacteria 10 can be reduced. The corrosion product 800 can be easily collected by disposing the product permeable body 900.

As described above, in the method for culturing the hydrogen-oxidizing bacteria 10 according to the second embodiment, the product permeable body 900 is disposed below the metal body 300 in contact with the liquid medium 200 in a gravity direction, the corrosion product 800 generated by the corrosion reaction passes through the product permeable body 900, and at least one of accommodation and collection of the corrosion product 800 is performed below the product permeable body 900 in the gravity direction.

According to this method, the corrosion product 800 is accommodated and collected by using the product permeable body 900, and therefore, the contact between the hydrogen-oxidizing bacteria 10 and the corrosion product 800 can be prevented, and the influence on the culture of the hydrogen-oxidizing bacteria 10 can be reduced.

In addition, the culture vessel 100 of the culturing apparatus 1000A for the hydrogen-oxidizing bacteria 10 according to the second embodiment includes the product permeable body 900 that allows the corrosion product 800 generated by the corrosion reaction to pass below the metal body 300 in the gravity direction. According to this configuration, the corrosion product 800 is accommodated and collected by using the product permeable body 900, and therefore, the contact between the hydrogen-oxidizing bacteria 10 and the corrosion product 800 can be prevented, and the influence on the culture of the hydrogen-oxidizing bacteria 10 can be reduced.

Hereinafter, modifications of the above-described embodiment will be described.

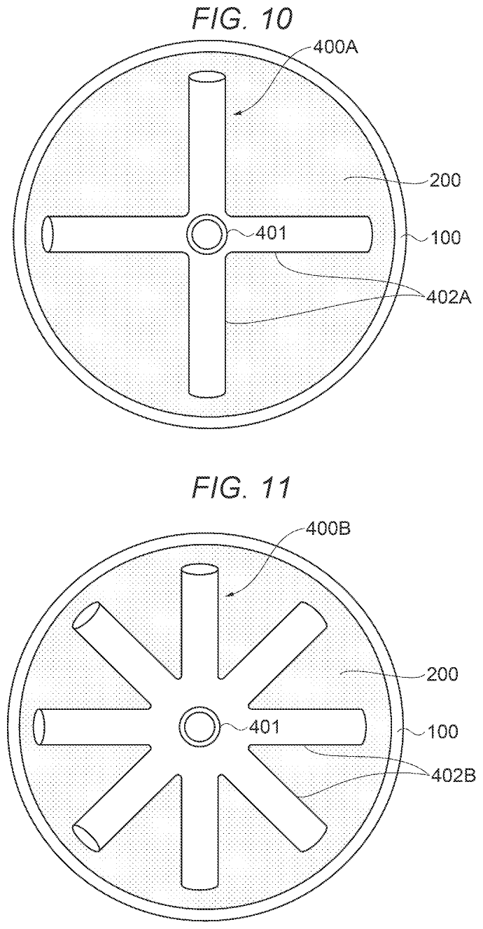

As described above, the nozzle 400 is not limited to being implemented by the second pipe 402 having the plurality of opening holes 410, and may be configured as shown in FIGS. 10 and 11. As shown in FIG. 10, a nozzle 400A in the modification is provided such that two pipes 402A intersect each other when the culture vessel 100 is viewed from above. An angle between the two pipes 402A may be uniform or different. In addition, each pipe 402A is provided with the plurality of opening holes 410 as described above. Also in this case, the culture vessel 100 may be rotated, or the nozzle 400A may be rotated.

As shown in FIG. 11, a nozzle 400B in the modification is provided such that the pipes 402B have a radial shape when the culture vessel 100 is viewed from above. As described above, each of a plurality of pipes 402B is provided with the plurality of opening holes 410. Also in this case, the culture vessel 100 may be rotated, or the nozzle 400B may be rotated.

In the culturing apparatus 1000 for the hydrogen-oxidizing bacteria 10 according to the present embodiment, the nozzle 400 preferably includes a plurality of nozzles 400 supplying the gas 600 to a plurality of locations of the liquid surface 201. According to this configuration, the plurality of nozzles 400 are provided, and therefore, the gas 600 can be blown to a plurality of locations of the liquid surface 201, and the liquid surface 201 can be rippled. Therefore, the gas 600 can be supplied into the rippled liquid medium 200.

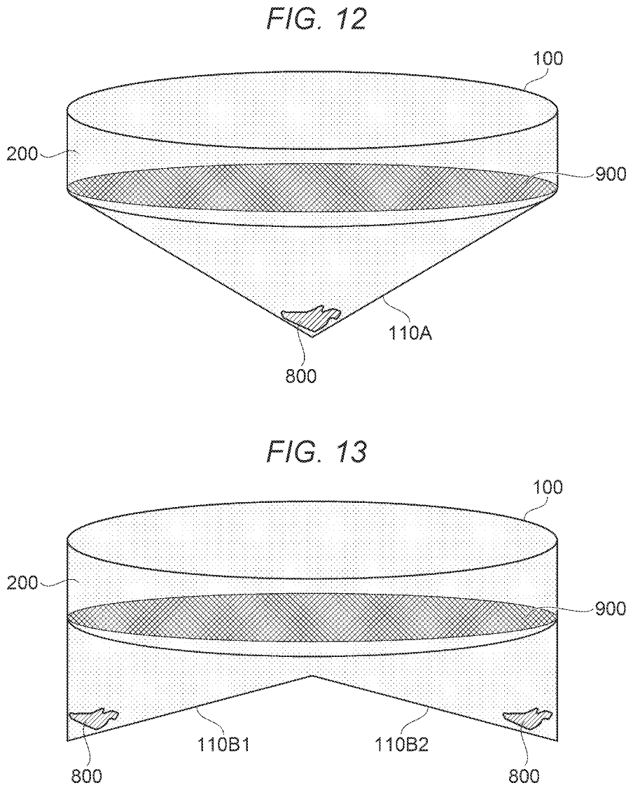

As described above, it is not limited that the bottom portion 100a of the planar culture vessel 100 is below the product permeable body 900, and configurations as shown in FIGS. 12 to 15 may be used. As shown in FIG. 12, a lower part of the culture vessel 100 in a modification is provided with an inverted conical accommodation portion 110A in which the space is gradually narrowed from the bottom of the culture vessel 100. That is, bottom structures having different depths are provided. With such a configuration, the corrosion product 800 that permeates the product permeable body 900 can be accommodated in only a part thereof, and the corrosion product 800 can be easily collected.

As shown in FIG. 13, a lower side of the culture vessel 100 in a modification is provided with two accommodation portions 110B1 and 110B2 at parts facing each other across a center of the circular culture vessel 100. The shapes of the accommodation portions 110B1 and 110B2 are not particularly limited as long as they are shapes by which the corrosion product 800 can be collectively accommodated.

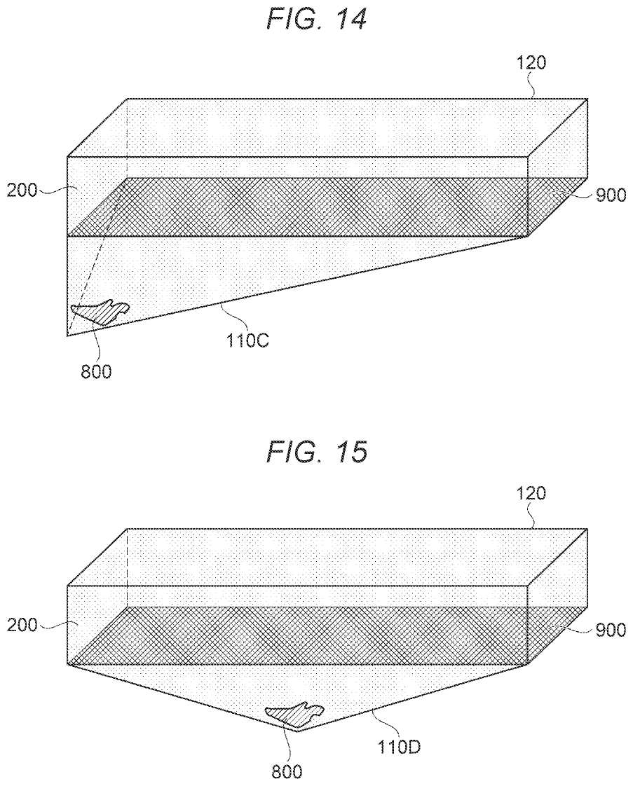

As shown in FIG. 14, a lower side of a rectangular culture vessel 120 in a modification is provided with, for example, an accommodation portion 110C at one corner among four corners of the culture vessel 120.

As shown in FIG. 15, a lower side of the culture vessel 120 in a modification is provided with, for example, an accommodation portion 110D at a center of the rectangular culture vessel 120.

As described above, in the culturing apparatus 1000 for the hydrogen-oxidizing bacteria 10 in the modifications, the culture vessel 100 preferably has bottom structures having different depths below the product permeable body 900. According to this configuration, bottom structures having different depths are provided, and therefore, the corrosion product 800 that has passed through the product permeable body 900 can be collected at the deepest part. Accordingly, the corrosion product 800 can be easily collected thereafter.

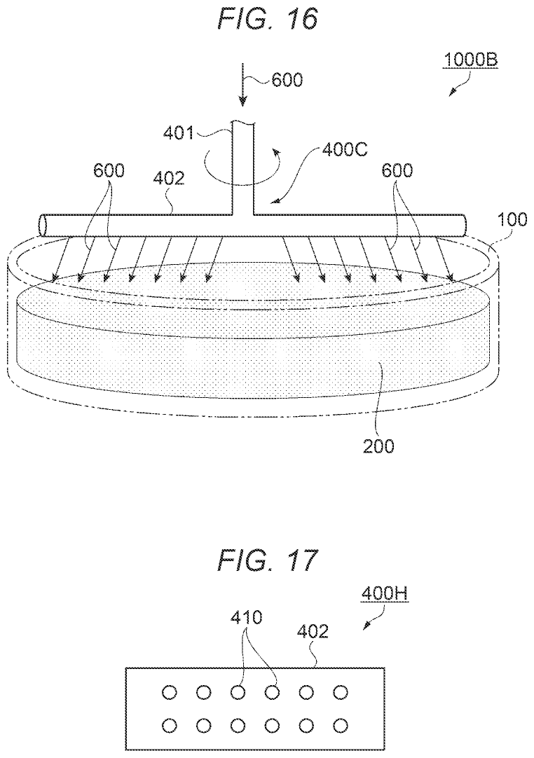

The gas 600 is not limited to being blown in the vertical direction from the second pipe 402 of the nozzle 400 as described above, and a configuration shown in FIG. 16 may be used. As shown in FIG. 16, in a culturing apparatus 1000B in a modification, with respect to the first pipe 401, the opening holes 410 are disposed on one side of the second pipe 402 such that the gas 600 is obliquely blown downward, and the opening holes 410 are disposed on the other side of the second pipe 402 such that the gas 600 is blown downward opposite to the above-described downward direction. That is, a nozzle 400C rotates around the first pipe 401 only by blowing the gas 600. According to this configuration, the gas 600 can be blown to the entire liquid medium 200 in the culture vessel 100 without mechanically rotating the nozzle 400C.

The nozzle 400 is not limited to the configuration in which the opening holes 410 are disposed side by side in a row in the second pipe 402, and may have a configuration shown in FIG. 17. As shown in FIG. 17, in a nozzle 400H in a modification, the opening holes 410 are disposed side by side in two rows in the second pipe 402. The opening holes 410 are not limited to being disposed side by side in two rows, and may be disposed in two or more rows.

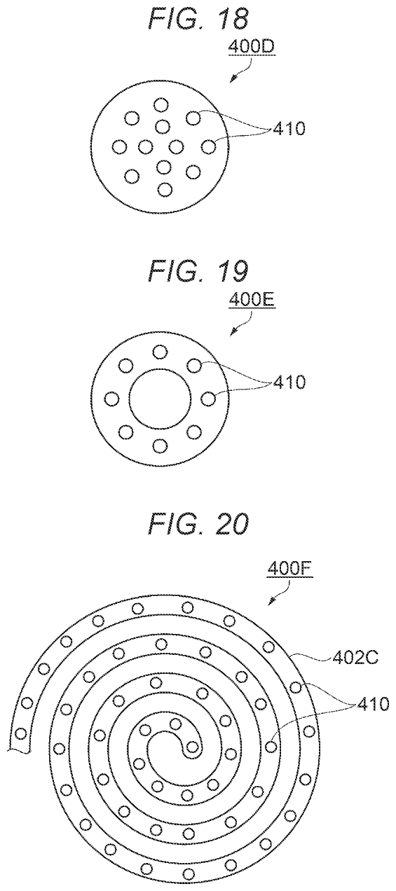

The opening holes 410 are not limited to being provided in a row shape in the second pipe 402 as described above, and for example, a configuration as shown in FIG. 18 may be used. A nozzle 400D in a modification shown in FIG. 18 has a surface parallel to the liquid surface 201 of the liquid medium 200, and the plurality of opening holes 410 are provided in this surface. In other words, the nozzle 400D has a shower head shape. The opening holes 410 may be disposed regularly or randomly. A size of the nozzle 400D may be substantially the same as a size of the liquid medium 200, or may be smaller than the size of the liquid medium 200 when the nozzle 400D is moved.

Instead of the nozzle 400D shown in FIG. 18, the opening holes 410 may be disposed in a row along an outer periphery of a circular nozzle 400E as shown in FIG. 19.

Instead of the nozzles 400D and 400E shown in FIGS. 18 and 19, a nozzle 400F shown in FIG. 20 may be used. As shown in FIG. 20, in the nozzle 400F in a modification, a pipe 402C is formed in a spiral shape, and the opening holes 410 are provided along an extending direction of the pipe 402C.

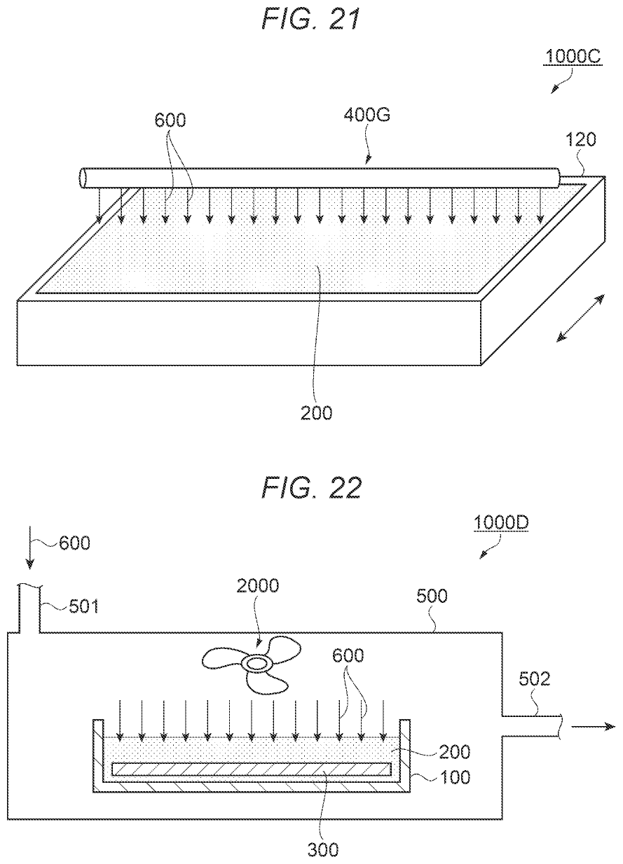

The culture vessel is not limited to being the circular culture vessel 100, and may be the rectangular culture vessel 120 as shown in FIG. 21. As shown in FIG. 21, a culturing apparatus 1000C in a modification includes the rectangular culture vessel 120 and a nozzle 400G having the same length as one side of the culture vessel 120. The culture vessel 120 may be moved in a direction intersecting an extending direction of the nozzle 400G, the nozzle 400G may be moved, or the two may be moved relative to each other.

As shown in FIG. 22, a fan 2000 may be disposed instead of the nozzle 400 in the chamber 500. In a culturing apparatus 1000D in a modification shown in FIG. 22, the fan 2000 is disposed above the culture vessel 100, specifically, above the liquid medium 200. In addition, the chamber 500 is provided with a supply port 501 for supplying the gas 600 and a discharge port 502 for discharging the gas 600. In this case, the liquid surface 201 of the liquid medium 200 can be rippled by the fan 2000.

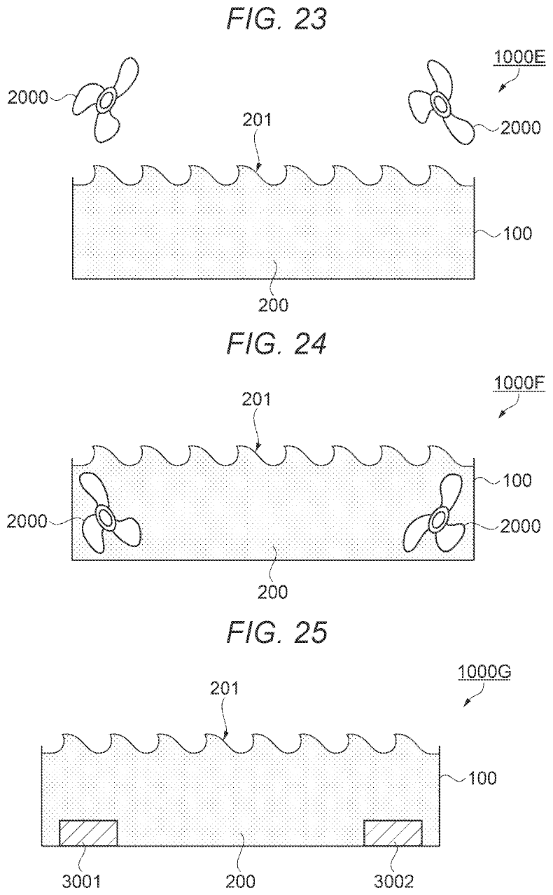

Two fans 2000 may be disposed above the culture vessel 100 as in a culturing apparatus 1000E in a modification shown in FIG. 23 instead of the above-described culturing apparatus 1000D. In addition, as in a culturing apparatus 1000F in a modification shown in FIG. 24, the fan 2000 may be disposed in the liquid medium 200 in the culture vessel 100 so that the liquid surface 201 is rippled.

As in a culturing apparatus 1000G in a modification shown in FIG. 25, quartz crystal vibrators 3001 and 3002 may be disposed in the culture vessel 100, that is, in the liquid medium 200, and the liquid medium 200 may be vibrated to cause the liquid surface 201 to ripple.

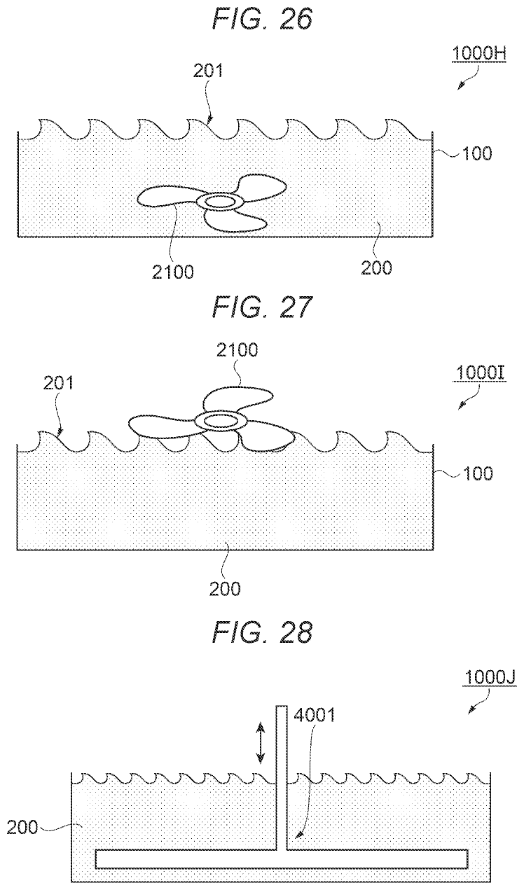

As in a culturing apparatus 1000H in a modification shown in FIG. 26, an impeller 2100 may be disposed in the liquid medium 200 so that the liquid surface 201 is rippled. In addition, as in a culturing apparatus 1000I in a modification shown in FIG. 27, the impeller 2100 may be disposed in a vicinity of the liquid surface 201 of the liquid medium 200 so that the liquid surface 201 is rippled.

As in a culturing apparatus 1000J in a modification shown in FIG. 28, a wave generator 4001 which is a plate-shaped baffle plate may be disposed in the liquid medium 200, and the liquid surface 201 may be rippled by moving the wave generator 4001 up and down.

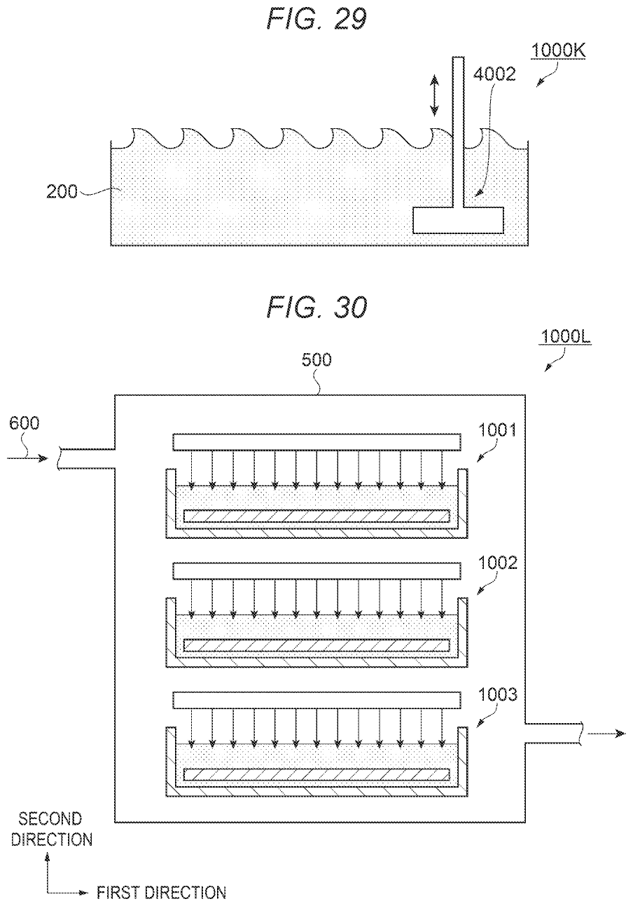

As in a culturing apparatus 1000K in a modification shown in FIG. 29, a wave generator 4002 which is a plate-shaped baffle plate may be disposed in a direction of one side of the culture vessel 100, and the liquid surface 201 may be rippled by moving the wave generator 4002 up and down.

As in a culturing apparatus 1000L in a modification shown in FIG. 30, culturing apparatuses 1001, 1002, and 1003 may be disposed in multiple stages in a second direction in the chamber 500. According to this configuration, the arrangement locations may be sequentially changed or may be sequentially replaced depending on a culture state in the culturing apparatuses 1001, 1002, and 1003.

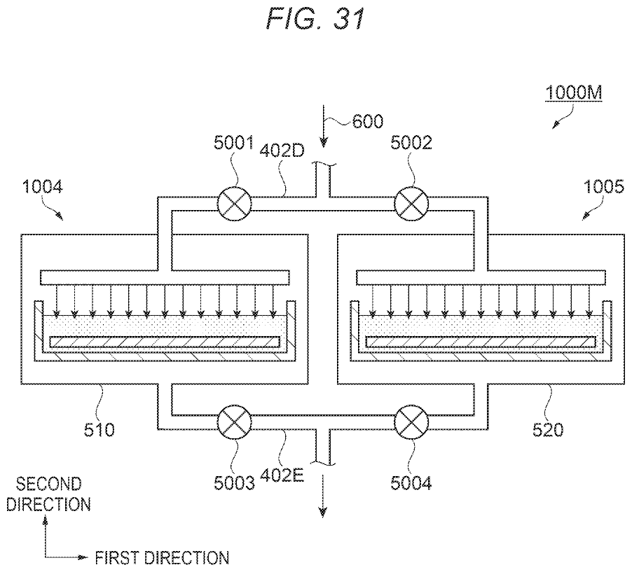

As in a culturing apparatus 1000M in a modification shown in FIG. 31, a culturing apparatus 1004 accommodated in a first chamber 510 and a culturing apparatus 1005 accommodated in a second chamber 520 may be provided in the first direction. The first chamber 510 and the second chamber 520 are coupled to each other by a pipe 402D via valves 5001 and 5002 on a supply side of the gas 600. The first chamber 510 and the second chamber 520 are coupled to each other by a pipe 402E via valves 5003 and 5004 on a discharging side of the gas 600. According to this configuration, for example, when the gas 600 generated in a thermal power station is effectively used, continuous operation can be performed while switching between the culturing apparatus 1004 to be operated and the culturing apparatus 1005 to be maintained.

As described above, in the culturing apparatus 1000M for the hydrogen-oxidizing bacteria 10 in the modification, it is preferable that the culturing apparatus 1000M includes the first culturing apparatus 1004 and the second culturing apparatus 1005, and the first culturing apparatus 1004 and the second culturing apparatus 1005 are disposed side by side in at least one of the first direction and the second direction intersecting the first direction. According to this configuration, the plurality of culturing apparatuses 1001 to 1005 are disposed, and therefore, for example, culture can be efficiently performed while performing switching such as performing different processes for each culturing apparatus.

In addition, the inside of the culture vessel 100 is not limited to a straight shape without unevenness, and for example, unevenness may be provided in a portion to be in contact with the liquid medium 200 to generate turbulence in the liquid medium 200.

What is claimed is:

1. A hydrogen-oxidizing bacteria culturing method comprising:
supplying a gas containing carbon dioxide to a liquid surface of a liquid medium inoculated with hydrogen-oxidizing bacteria that are growable using the carbon dioxide as a carbon source; and
supplying hydrogen to the hydrogen-oxidizing bacteria, the hydrogen being generated by bringing the liquid medium into contact with a metal body and causing a corrosion reaction in the metal body.

2. The hydrogen-oxidizing bacteria culturing method according to claim 1, wherein
in the supplying the gas, the gas is blown at a pressure at which the liquid surface of the liquid medium is ripplable.

3. The hydrogen-oxidizing bacteria culturing method according to claim 1, wherein
in the supplying the gas, the gas is blown to portions corresponding to at least a plurality of locations of the liquid surface.

4. The hydrogen-oxidizing bacteria culturing method according to claim 1, wherein
in the supplying the gas, the liquid medium and the supplied gas are moved relative to each other.

5. The hydrogen-oxidizing bacteria culturing method according to claim 1, wherein
the metal body contains a metal element having an ionization tendency higher than an ionization tendency of the hydrogen.

6. The hydrogen-oxidizing bacteria culturing method according to claim 5, wherein
the metal element is Ca, Mg, Al, Ti, or Zn.

7. The hydrogen-oxidizing bacteria culturing method according to claim 1, wherein
the metal body contains a magnesium-based alloy or a composite material containing a magnesium-based alloy.

8. The hydrogen-oxidizing bacteria culturing method according to claim 1, wherein
the supplying the hydrogen to the hydrogen-oxidizing bacteria includes an operation of changing at least one of a temperature and a pH of the liquid medium, a flow rate of the liquid medium, and a moving speed of the metal body relative to the liquid medium.

9. The hydrogen-oxidizing bacteria culturing method according to claim 1, wherein
a product permeable body is disposed below the metal body in contact with the liquid medium in a gravity direction, a corrosion product generated by the corrosion reaction passes through the product permeable body, and at least one of accommodation and collection of the corrosion product is performed below the product permeable body in the gravity direction.

10. A hydrogen-oxidizing bacteria culturing apparatus comprising:
a culture vessel;
a liquid medium accommodated in the culture vessel and to be inoculated with hydrogen-oxidizing bacteria;
a metal body configured to generate hydrogen by causing a corrosion reaction by contact with the liquid medium; and
a nozzle disposed to face a liquid surface of the liquid medium and configured to supply a gas containing carbon dioxide to the liquid surface.

11. The hydrogen-oxidizing bacteria culturing apparatus according to claim 10, wherein
the nozzle includes a plurality of nozzles configured to supply the gas to a plurality of locations of the liquid surface.

12. The hydrogen-oxidizing bacteria culturing apparatus according to claim 10, wherein
the culture vessel and the nozzle are moved relative to each other.

13. The hydrogen-oxidizing bacteria culturing apparatus according to claim 10, wherein
the culture vessel includes a product permeable body through which a corrosion product generated by the corrosion reaction passes below the metal body in a gravity direction.

14. The hydrogen-oxidizing bacteria culturing apparatus according to claim 13, wherein
the culture vessel has, below the product permeable body, a bottom structure having different depths.

15. The hydrogen-oxidizing bacteria culturing apparatus according to claim 10, wherein
the culturing apparatus includes a first culturing apparatus and a second culturing apparatus, and
the first culturing apparatus and the second culturing apparatus are disposed side by side in at least one of a first direction and a second direction intersecting the first direction.
